(19) United States
(12) Patent Application Publication
(10) Pub. No.: US 2019/0307963 A1
(43) Pub. Date: Oct. 10, 2019

(54) SYRINGE ASSEMBLY WITH RELEASABLE BACKSTOP AND METHOD OF USE

(71) Applicant: Fisher Clinical Services GmbH, Allschwil (CH)

(72) Inventors: Patrick Locati, Ettingen (CH); Guido Hunkeler, Schonenbuch (CH)

(21) Appl. No.: 15/949,718

(22) Filed: Apr. 10, 2018

(51) Int. Cl. A61M 5/315

(52) U.S. Cl. CPC A61M 5/31501; A61M 2005/31508

(57) ABSTRACT

A syringe assembly includes a syringe barrel that bounds a compartment extending between a proximal end and an opposing distal end. A stopper is movably disposed with the compartment of the syringe barrel. A plunger rod includes a shaft having a proximal end and an opposing distal end and a plurality of teeth disposed on the shaft, the distal end of the shaft being secured to the stopper. A backstop includes a base coupled to the syringe barrel; a first wing and a second wing secured to the base so as to be at least partially disposed on opposing sides of the base; and a first arm projecting from the first wing. The first wing is movable between a first position where an engaging portion of the first arm engages with the plunger rod and a second position wherein the first arm is spaced apart from the plunger rod.

### CROSS-REFERENCE TO RELATED APPLICATIONS

Not applicable.

### BACKGROUND OF THE INVENTION

### 1. The Field of the Invention

The present invention relates to syringe assemblies having a plunger rod backstop and related methods of use.

### 2. The Relevant Technology

Clinical trials for some pharmaceutical drugs require that the drug dosage be delivered by injection through the use of a syringe. As part of the clinical trial protocols, the syringes are preloaded with a single dose of the drug or a placebo prior to shipping and use. The plunger rod for each syringe is typically attached to a stopper within the syringe barrel prior to shipping. Precautions are taken to ensure that there is no excessive movement of the stopper relative to the syringe barrel during transport of the preloaded syringes. Excessive movement of the stopper relative to the syringe barrel can potentially result in contamination of the dosage.

Movement of the stopper during transport can be a result of a change in atmospheric pressure. For example, the portion of the syringe barrel containing the dosage will also typically contain an air bubble. If the preloaded syringes are being transported by aircraft, the decrease in atmospheric pressure during the flight can cause the gas bubble to expand. In turn, expansion of the gas bubble can result in movement of stopper which, in turn, can potentially result in the dosage being contaminated.

In one attempt to preclude movement of the stopper during transport of the preloaded syringes, each preloaded syringe is housed within a separate, specially designed packaging case. The packaging case holds the plunger rod fixed relative to the syringe barrel so that neither the plunger rod nor the stopper can move relative to the syringe barrel when the preloaded syringe is subject to a change in atmospheric pressure.

Although the packaging cases are useful in preventing unwanted movement of the plunger rod and stopper, they have a number of shortcomings. For example, the packing cases are relatively large and must be reinforced to withstanding the applied pressures. As such, the packaging cases are a significant expense to produce, transport and store. Furthermore, the packaging cases are designed for a specific syringe configuration having a specific dose size. Accordingly, different packaging cases must be made for different syringes and for different dose sizes. This requirement adds to the cost and complexity of packaging and distribution of the preloaded syringes.

One approach to solving some of the above shortcomings is disclosed in US Patent Publication No. 2016/0144122 to Locati et al. The Locati publication discloses a syringe assembly that includes a syringe having a backstop attached thereto. The backstop only permits the plunger rod to move distally into the syringe barrel and not proximally. Although the disclosed syringe assembly achieves some benefits, it has other shortcomings. For example, the syringe assembly of Locati incorporates a ratchet to control movement of the plunger rod. The ratchet, however, creates a noise during operation that can annoy or potentially scare a patient when receiving a shot. The ratchet also requires an additional force to overcome the ratchet mechanism when first depressing the plunger. As such, it is difficult to dispense the dosage within the syringe assembly in an easy and smooth motion. Furthermore, the syringe assembly of Locati publication prevents all ability to partially withdraw the plunger rod from the syringe barrel which can be desirable in some situations. Other shortcomings also exist.

Accordingly, what is needed in the art are syringe assemblies that overcome all or some of the above shortcomings.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a front perspective view of an inventive syringe assembly incorporating features of the present invention;

FIG. 2 is a partially exploded view of the syringe assembly depicted in FIG. 1;

FIG. 3 is a perspective view of the stopper of the syringe assembly depicted in FIG. 1;

FIG. 4 rear perspective view of syringe assembly depicted in FIG. 1;

FIG. 5 is a bottom perspective view of the backstop of the syringe assembly depicted in FIG. 1;

FIG. 6 is a left side perspective view of the backstop depicted in FIG. 5;

FIG. 7 is a top perspective view of the backstop depicted in FIG. 5;

FIG. 8 is a front elevational view of the backstop depicted in FIG. 5;

FIG. 9 is a bottom plan view of the backstop depicted in FIG. 5;

FIG. 10 is a front elevational view of an upper end of the syringe assembly depicted in FIG. 1 with the wings of the backstop in a first position;

FIG. 11 is a front elevational view of an upper end of the syringe assembly depicted in FIG. 10 with the wings of the backstop in a second position;

FIG. 12 is a perspective view of the syringe assembly depicted in FIG. 1 being operated by the hand of a user; and

FIG. 13 is a perspective view of an alternative embodiment of the syringe assembly depicted in FIG. 1 having a modified plunger rod.

### DETAILED DESCRIPTION OF THE PREFERRED EMBODIMENTS

Before describing the present disclosure in detail, it is to be understood that this disclosure is not limited to particularly exemplified apparatus, systems, methods, or process parameters that may, of course, vary. It is also to be understood that the terminology used herein is only for the purpose of describing particular embodiments of the present invention, and is not intended to limit the scope of the invention in any manner.

All publications, patents, and patent applications cited herein, whether supra or infra, are hereby incorporated by reference in their entirety to the same extent as if each individual publication, patent, or patent application was specifically and individually indicated to be incorporated by reference.

The term “comprising” which is synonymous with “including,” “containing,” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “port” includes one, two, or more ports.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the invention or claims.

Where possible, like numbering of elements have been used in various figures. Furthermore, multiple instances of an element and or sub-elements of a parent element may each include separate letters appended to the element number. For example two instances of a particular element “88” may be labeled as “88A” and “88B”. In that case, the element label may be used without an appended letter (e.g., “88”) to generally refer to instances of the element or any one of the elements. Element labels including an appended letter (e.g., “88A”) can be used to refer to a specific instance of the element or to distinguish or draw attention to multiple uses of the element. Furthermore, an element label with an appended letter can be used to designate an alternative design, structure, function, implementation, and/or embodiment of an element or feature without an appended letter. Likewise, an element label with an appended letter can be used to indicate a sub-element of a parent element. For instance, an element “12” can comprise sub-elements “12A” and “12B.”

Various aspects of the present devices and systems may be illustrated by describing components that are coupled, attached, secured and/or joined together. As used herein, the terms “coupled”, “attached”, “secured” and/or “joined” are used to indicate either a direct connection between two components or, where appropriate, an indirect connection to one another through intervening or intermediate components. In contrast, when a component is referred to as being “directly coupled”, “directly attached”, “directly secured” and/or “directly joined” to another component, there are no intervening elements present. Furthermore, as used herein, the terms “connection,” “connected,” and the like do not necessarily imply direct contact between the two or more elements.

Various aspects of the present devices, systems, and methods may be illustrated with reference to one or more examplary embodiments. As used herein, the term “embodiment” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

In general, the present invention is directed to syringe assemblies that are designed to hold a dosage. The syringe assemblies are configured so that when they are subject to a change in pressure, such as when being transported by aircraft, the plunger rod is restrained from retracting out of the syringe barrel. However, the plunger rod can still be manually depressed when it is desired to dispense the dosage from the syringe barrel. In one embodiment, the plunger rod can be selectively advanced into or partially retract out of the syringe barrel without mechanical interference.

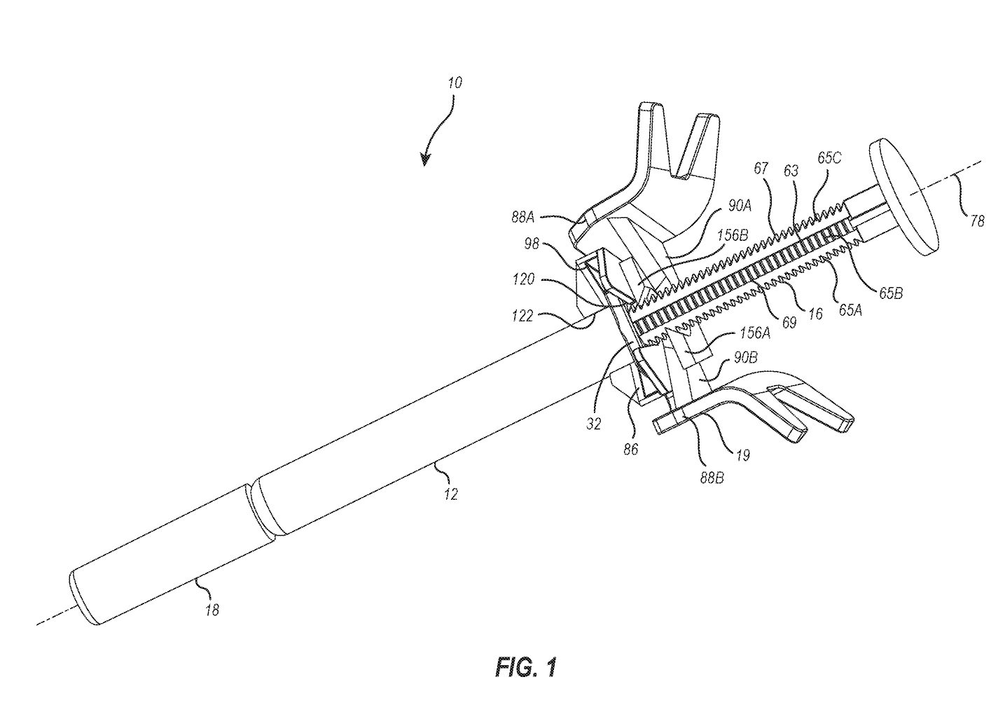

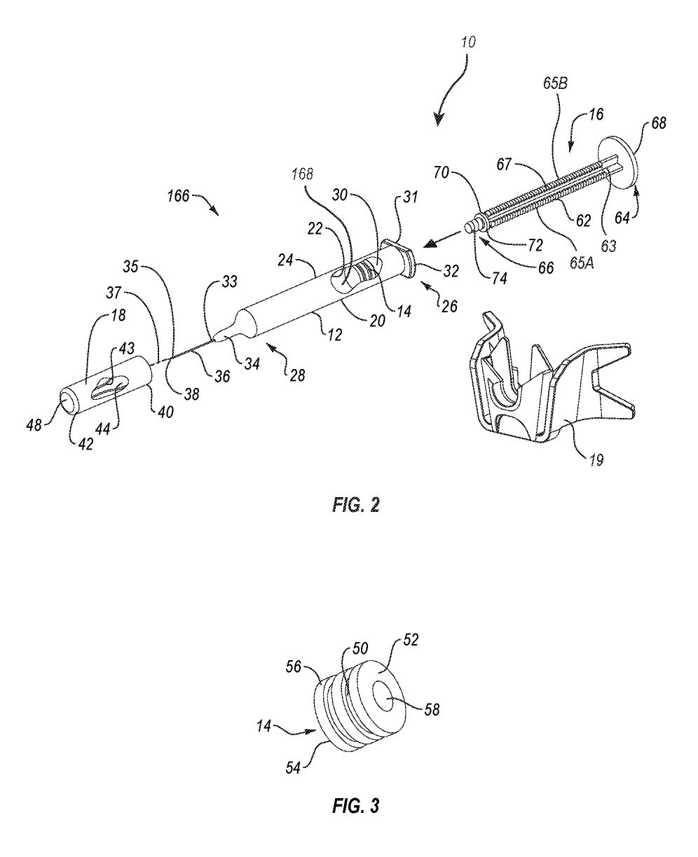

Depicted in FIGS. 1 and 2 is one embodiment of a syringe assembly 10 incorporating features of the present invention. In general, syringe assembly 10 comprises a syringe barrel 12, a needle 36 extending from a distal end of syringe barrel 12, a needle shield 18 that covers needle 36, a stopper 14 that is received within syringe barrel 12, a plunger rod 16 that couples with stopper 14, and a backstop 19 that selectively restrains movement of plunger rod 16 relative to syringe barrel 12. The above elements will now be discussed in greater detail.

As depicted in FIG. 2, syringe barrel 12 comprises an elongated cylindrical body 20 having an interior surface 22 and an exterior surface 24 that longitudinally extend between a proximal end 26 and an opposing distal end 28. Interior surface 22 bounds a compartment 30 that extends between opposing ends 26 and 28. An opening 31 is formed at proximal end 26 and communicates with compartment 30. A flange 32 radially outwardly projects from proximal end 26. Flange 32 can encircle proximal end 26 or can project out from opposing sides of proximal end 26.

Projecting from body 20 at distal end 28 is an elongated stem 34. Stem 34 has a transverse cross section with a diameter that is smaller than the diameter of the transverse cross section of body 20. As discussed below, stem 34 is configured to receive and engage needle shield 18. Syringe barrel 12, or at least body 20, is typically made of a transparent or semi-transparent material such as clear plastic or glass. In other embodiments, however, body 20 can be opaque.

Projecting from stem 34 of syringe barrel 12 along a central longitudinal axis 37 of body 20 is tubular needle 36. Needle 36 has a proximal end 33 that communicates with compartment 30 of syringe barrel 12 and an opposing distal end 35 that terminates at a sharpened tip 38. Needle 36 is typically made of metal. In the embodiment depicted, needle 36 is permanently attached to syringe barrel 12 such as by being molded into stem 34 or by being attached by an adhesive. In alternative embodiments, needle 36 or the combination of needle 36 and stem 34 can be removably and/or mechanically attached to syringe barrel 12. For example, stem 34 and needle 36 can be formed with complementary halves of a luer-lock or snap-fit connection that permit fluid coupling therebetween. Other types of fluid connections can also be used.

As also depicted in FIG. 2, needle shield 18 has a first end 40 designed for receiving needle 36 and stem 34 of syringe barrel 12 and an opposing second end 42 that terminates at a terminal end face 48. Needle shield 18 has an interior surface 43 that bounds a cavity 44 that is open at first end 40. Cavity 44 is configured to receive needle 36 and stem 34 so that a friction tight engagement is formed between interior surface 43 and stem 34. When needle shield 18 is attached to syringe barrel 12 by the friction tight engagement, needle 36 is safely enclosed within cavity 44. Needle shield 18 typically has an outer portion comprised of a rigid plastic that prevents needle 36 from puncturing therethrough and has a more flexible or elastomeric inner portion that engages with stem 34. In alternative embodiments, a mechanical connection can be used to secure needle shield 18 to syringe barrel 12. For example, needle shield 18 and stem 34 can be formed with complementary halves of a luer-lock connection that permit coupling therebetween. Other types of connection can also be used.

Slidably disposed within compartment 30 of syringe barrel 12 is stopper 14. As depicted in FIG. 3, stopper 14 has an annular side face 50 that extends between a proximal end face 52 and an opposing distal end face 54. Encircling and radially outwardly projecting from side face 50 are a plurality of lip seals 56. Lip seals 56 maintain a liquid tight seal against interior surface 22 (FIG. 2) of syringe barrel 12 as stopper 14 longitudinally moves within compartment 30. Accordingly, as stopper 14 advances towards distal end 28 of syringe barrel 12, any liquid within compartment 30 is dispensed out through needle 36. Centrally formed on proximal end face 52 is a threaded socket 58. Stopper 14 is typically comprised of an elastomeric material that enables a slidable, sealed engagement between stopper 14 and syringe barrel 12. Other materials that will achieve the desired functional operation can also be used.

Returning to FIG. 2, plunger rod 16 comprises an elongated shaft 62 that extends between a proximal end 64 and an opposing distal end 66. As depicted in FIGS. 1, 2 and 4, shaft 62 has a “+” shaped transverse cross section that includes a central core 63 having four, elongated, radially spaced apart ribs 65A, 65B, 65C and 65D radially outwardly projecting from core 63 and extending between proximal end 64 and an opposing distal end 66. Each adjacent pair of ribs 65 are separated by an elongated slot 69. Each rib 65 has an outer edge with a plurality of spaced apart teeth 67 formed along the length thereof. Shaft 62 has a configuration that better enables mechanically grasping plunger rod 16. This mechanical grasping of plunger rod 16 is used during the attachment of plunger rod 16 to stopper 14 as described in US Patent Publication No. 2016/0038685, published Feb. 11, 2016, which is incorporated herein by specific reference. In different embodiments, there can be two, three, five, or more radially spaced apart ribs 65 extending along the length of shaft 62.

As depicted in FIG. 10, each tooth 67 has an asymmetrical configuration that includes a proximal face 80 and a distal face 82 that intersect at an outside corner 84. Proximal face 80 is typically flat forming an inside angle θ₁ relative to a central longitudinal axis 78 of shaft 62 that is or is about 90°. In other embodiments, however, proximal face 80 could inwardly taper so that angle θ₁ is less than 90°, such as in a range between 70° and 90°. In still other embodiments, proximal face 80 could outwardly taper slightly such as in range between 90° and 110°. Other angles can also be used.

Distal face 82 is sloped forming an inside angle θ₂ relative to axis 78 that is typically in a range between about 20° and 80° with between 40° and 70° being more common. Again, other angles can be used. Typically there are more than 20 longitudinally spaced teeth 67 and more common more than 30, 40, or 60 teeth 67 of each rib 65. In other embodiments, less than 20 teeth 67 can be used. Teeth 67 can butt directly against each other, e.g., distal face 82 of one tooth 67 can intersect with proximal face 80 of the adjacent tooth 67. In other embodiments, a gap can be formed between adjacent teeth. The gap is the longitudinal distance between where distal face 82 ends and the adjacent proximal face 80 starts. Any gap, however, is typically small, such as less than 2 mm and more commonly less than 1 mm or less than 0.5 mm. As a result of the tapering of distal face 82 in combination with any gap, a notch 87 is formed between each pair of adjacent teeth 67.

Returning to FIG. 2, disposed at proximal end 64 of shaft 62 is a thumb rest 68. Formed at distal end 66 of shaft 62 is an end plate 70. End plate 70 has a distal end face 72 from which a threaded stem 74 centrally projects. Threaded stem 74 is configured to thread into socket 58 of stopper 14 (FIG. 3) so that stopper 14 is securely connected to plunger rod 16 and so that distal end face 72 of plunger rod 16 can sit flush against proximal end face 52 of stopper 14. Plunger rod 16 is typically molded from a polymeric material, but other materials can also be used.

Returning to FIG. 1, backstop 19 is removably coupled with syringe barrel 12 and is used for selectively controlling the movement of plunger rod 16. In general, backstop 19 comprises a base 86 having a first wing 88A and an opposing second wing 88B secured to base 86 so as to be at least partially disposed on opposing sides of base 86. Arms 90A and 90B inwardly project from wings 88A and 88B, respectfully, and engage plunger rod 16 to control the movement thereof. The parts of backstop 19 will now be discussed in greater detail.

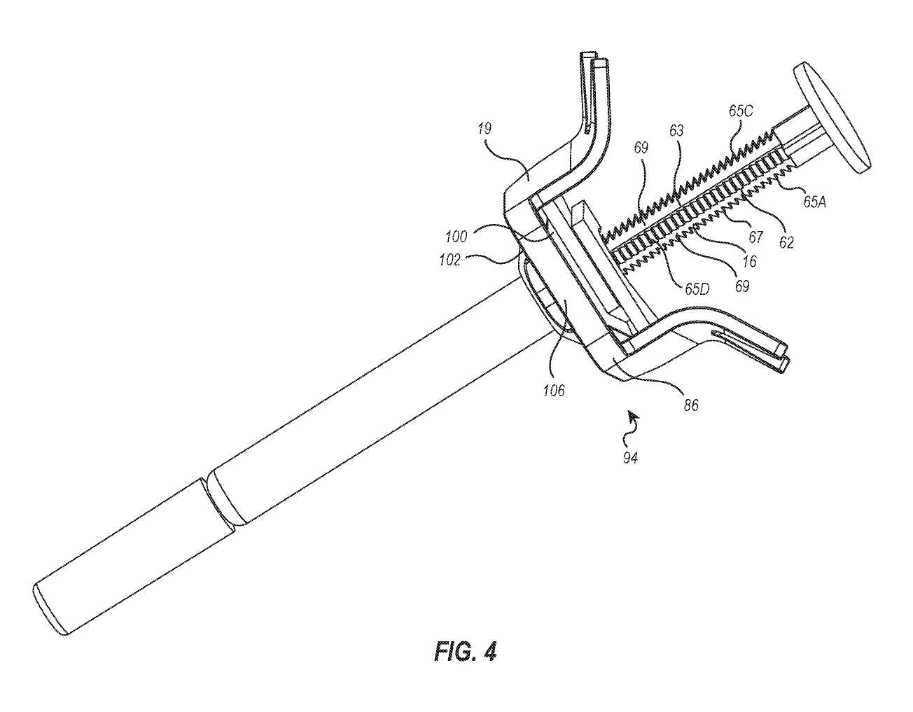

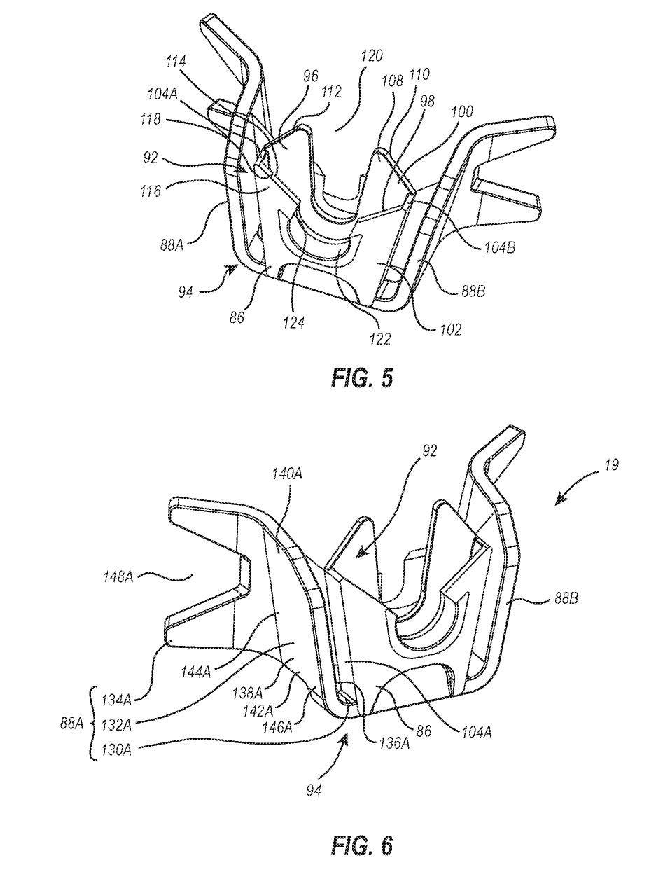

Turing to FIG. 5, base 86 has a front end 92 and an opposing back end 94. An opening 96 is formed at front end 92 that communicates with a compartment 98 formed within base 86. More specifically, base 86 comprises a top wall 100 and a bottom wall 102 that extend between front end 92 and back end 94 and that also extend laterally between opposing side walls 104A and 104B. As depicted in FIG. 4, a back wall 106 extends between top wall 100 and bottom wall 102 at back end 94. Returning to FIG. 5, top wall 100 has an interior face 108 and an opposing exterior face 110 that terminate at and end face 112 located at front end 92. Likewise, bottom wall 102 has an interior face 114 and an opposing exterior face 116 that terminate at and end face 118 located at front end 92. Compartment 98 is bounded between interior face 108 of top wall 100 and interior surface 114 of bottom wall 102. More specifically, compartment 98 is partially bounded by each of top wall 100, bottom wall 102, side walls 104A and B and back wall 106.

End face 112 of top wall 100 projects outward away from back end 94. A slot 120 is centrally recessed into end face 112 and extends toward back end 94. Slot 120 extends between interior face 108 and exterior face 110 of top wall 100 and has a U-shaped configuration. Slot 120 is sized to receive plunger rod 16. Although not required, in the depicted embodiment, slot 120 flares outward toward end face 112.

End face 118 of bottom wall 102 projects inward toward back end 94. A slot 122 is centrally recessed into end face 118 and extends toward back end 94. Slot 122 extends between interior face 114 and exterior face 116 of bottom wall 102, is aligned with sot 120, and has a generally U-shaped configuration. More specifically, slot 122 has a C-shaped configuration with a constricted mouth 124 and is configured to receive syringe barrel 12.

With reference to FIGS. 1 and 5, during use, flange 32 of syringe barrel 12 is slid within compartment 98 through opening 96 so that plunger rod 16 is received within slot 120 and syringe barrel 12 is received within slot 122. Mouth 124 is slightly smaller than the diameter of syringe barrel 12 so that as syringe barrel 12 is being pushed into slot 122, mouth 124 resiliently expands to allow syringe barrel 12 to pass through mouth 124 and into slot 122. Mouth 124 then resiliently closes around syringe barrel 12 to snugly secure syringe barrel 12 within slot 122, thereby removably securing syringe barrel 12 to base 86. In alternative embodiments, slot 122 need not have a C-shaped configuration but could have a U-shaped configuration or rounded V-shaped configuration that does not expand when syringe barrel 12 is received therein.

As depicted in FIG. 6, first wing 88A comprises a mounting portion 130A that is secured to base 86, a body portion 132A secured to mounting portion 130A, and a finger rest 134A projecting from body portion 132A. More specifically, body portion 132A has an inside face 136A and an opposing outside face 138A that both extend between a front end 140A and an opposing back end 142A and extend between an upper end 144A and an opposing lower end 146A. Body portion 132A is spaced apart from base 86 but extends along sidewall 104A of base 86 between front end 92 and back end 94 of base 86. As depicted, body portion 132A can be disposed parallel to sidewall 104A. In other embodiments, however, body portion 132A can slope inward toward front end 92 or outward away from front end 92 as it extends from back end 94 to front end 92. In the embodiment depicted, body portion 132A comprises a panel wherein inside face 136A and outside face 138A are flat and are disposed in parallel alignment. In other embodiments, inside face 136 and/or outside face 138 can be sloped or otherwise contoured and need not be disposed in parallel alignment.

Mounting portion 130A is shown extending from body portion 132A at lower end 146A and back end 142A to base 86 at back end 94. In one embodiment, as shown in FIG. 9, an inside angle θ₃ is formed between mounting portion 130A and body portion 132A that is in a range between 70° and 110° with between 80° and 100° being more common. Other angles can also be used. As discussed below in greater detail, mounting portion 130A is configured and positioned so that body portion 132A can move between different positions by flexing of mounting portion 130A.

Returning to FIG. 6, finger rest 134A outwardly projects from body portion 132A at upper end 144A so as to project away from base 86. In one embodiment, as depicted in FIG. 8, finger rest 134A can project body portion 132A so as to form an inside angle θ₄ that is in a range between 90° and 170° with between 100° and 160° and between 120° and 150° being more common. Other angles can also be used. In the embodiment depicted, a slot 148A is formed on and centrally extends through finger rest 134A. In other embodiments, slot 148A can be eliminated.

Wings 88B is the mirror image of wing 88A and has the same components and is attached in the same location to base 86 except on the opposite side of base 86. Accordingly, the above discussion and other discussions herein with regard to the shape and use of wing 88A and all alternatives and modifications thereto are also applicable to wing 88B. Furthermore, like element between wings 88A and 88B are identified herein by like reference characters except that the elements of wing 88B are identified with the letter “B”.

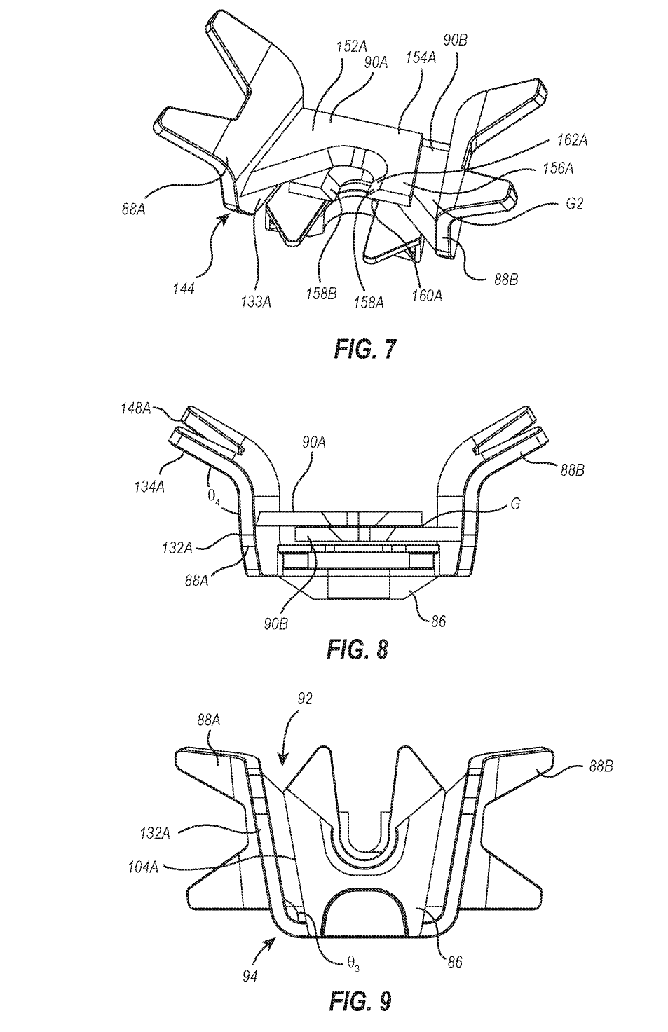

Turning to FIG. 7, arm 90A projects from wing 88A toward wing 88B while arm 90B projects from wing 88B toward wing 88A. Arms 88A and 88B are overlapping and are typically disposed in parallel alignment. That is, as previously discussed and depicted in FIG. 1, plunger rod 16 has a central longitudinal axis 78 extending along the length thereof. When central longitudinal axis 78 of plunger rod 16 is vertically disposed, first arm 90A and second arm 90B are overlapping with first arm 90A being vertically disposed above second arm 90B. In an alternative embodiment, second arm 90B could be disposed vertically above first arm 90A. As shown in FIG. 8, a slight gap G is formed between first arm 90A and second arm 90B so that arms 90A and 90B can freely move relative to each other.

Returning to FIG. 7, arm 90A typically outwardly projects from upper end 144 of body 133A of wing 88A. A gap G2 is formed between the terminal end of arm 90A and second wing 88B. First wing 88A has a generally L-shaped configuration that includes an elongated leg 152A that projects from wing 88A to a terminal end 154A. A foot 156A projects forward from terminal end 154A. Foot 156A has an inside edge that faces back toward first wing 88A and forms an engagement lip 158A. Engagement lip 158A has a wedge shaped cross section and is configured to be received within notches 87 of plunger rod 16 as shown in FIG. 10. In one embodiment, engagement lip 158A can have a shape complementary to notches 87. For example, engagement lip 158A has a distal face 160A and an opposing proximal face 162A. Distal face 160A is typically disposed in a plane perpendicular to longitudinal axis 78 of plunger rod 16 and is disposed parallel to distal face 80 of teeth 67. As such, when engagement lip 158A is received within a notch 87, distal face 160A of engagement lip 158A engages against proximal face 80 of a tooth 67 to prevent plunger rod 16 from retracting out of syringe barrel 12.

In contrast, proximal face 156A of engagement lip 158A is tapered or sloped relative to distal face 160A to enable plunge rod 16 to be selectively advanced onto syringe barrel 12. Specifically, proximal face 156A is typically disposed at an angle that is complementary to the angle, previously discussed above, at which distal face 82 of teeth 67 are disposed. As a result, when plunger rod 16 is pressed toward compartment 30 of syringe barrel 12, distal face 82 of a tooth 67 rides against and radially outwardly pushes proximal face 156A of engagement lip 158A of first arm 90A by resiliently inwardly flexing wing 88A. Distal face 82 of tooth 67 continues to ride against proximal face 156A of engagement lip 158A until outside corner 84 of the tooth 67 passes over engagement lip 158A. Flexed wing 88A then resiliently rebounds forcing engagement lip 158A back into the next notch 87. The process is then be repeated for the next tooth 67 until plunger rod 16 is advanced to the desired depth into compartment 30 (FIG. 1). Those skilled in the art will appreciate that proximal face 162A and distal face 82 can be formed in a variety of different angles and that the angles can be complementary or different from each other. Faces 162A and 82 simply need to be sloped so that teeth 67 radially outwardly press engagement lip 158A so that plunger rod 16 can advance into compartment 30.

Arm 90B is the mirror image of arm 90A and has the same components and is attached in the same location to wing 88B except on the opposite side of base 86 Like elements between arms 90A and 90B are identified herein by like reference characters except that the elements of arm 90B are identified with the letter “B”. Furthermore, wing 88B interacts with teeth 67 on plunger rod 16 in the same manner as wing 88A except that engagement lip 158B engages with teeth 67 on the opposite side of plunger rod 16. It is also appreciated that engagement lip 158B is moved in an opposite direction to engagement lip 158A as plunger rod 16 is advanced into syringe barrel 12. Accordingly, the above discussion and other discussions herein with regard to arm 90A and all alternatives and modifications thereto are also applicable to arm 90B.

Backstop 19 is typically molded from a polymeric material and is commonly formed as a single, unitary, integral structure. That is, base 86, wings 88 and arms 90 are simultaneously, integrally formed as a one piece member as opposed to initially being formed as separate members that are subsequently coupled together.

During assembly, a pre-loaded syringe barrel 166 is provided, as depicted in FIG. 2. The pre-loaded syringe barrel 166 comprises syringe barrel 12, needle shield 18 secured to distal end 28, stopper 14 disposed within compartment 30 of syringe barrel 12, and a liquid dosage 168 disposed within compartment 30 distal of stopper 14. Dosage 168 can comprise a drug, a drug comparative, or a placebo, such as a saline solution. Often, although not always, an air bubble can also be located with compartment 30 distal of stopper 14.

Once pre-loaded syringe barrel 166 is provided, plunger rod 16 can be advanced into compartment 30. Stem 74 of plunger rod 16 is then threaded into socket 58 (FIG. 3) of stopper 14 so as to secure stopper 14 to plunger rod 16. Care is taken to not substantially move stopper 14 within compartment 30 while stopper 14 is being secured to plunger rod 16 so as to avoid contamination of dosage 168. One example of how to attach plunger rod 16 to stopper 14 is disclosed in U.S. Pat. No. 9,545,480, issued Jan. 17, 2017, which is incorporated herein by specific reference. Other methods can also be used.

Once plunger rod 16 is secured to stopper 14, as depicted in FIG. 1, backstop 19 is secured to both syringe barrel 12 and plunger rod 16. As discussed above, this is accomplished by flange 32 of syringe barrel 12 being slid into compartment 98 of backstop 19 so that syringe barrel 12 is received within slot 122. A variety of different mechanisms can be used for securing together syringe barrel 12 and backstop 19. For example, the connection can be a result of frictional engagement between backstop 19 and flange 32 and/or syringe barrel 12. As previously discussed, syringe barrel 12 can also snap-fit into slot 122 of backstop 19. In an alternative embodiment, a retainer can be couple to backstop 19, such as by a snap-fit connection, so that the retainer extends over syringe barrel 12 and thus holds backstop 19 to syringe barrel 12. For example, the combined backstop 19 and retainer could completely encircle or substantially encircle syringe barrel 12. In a like embodiment, backstop 19 can be comprised of two halves that couple together around syringe barrel 12 and/or flange 32 so as to completely or substantially encircle syringe barrel 12. In other embodiments, backstop 19 can be attached to syringe barrel 12 and/or flange 32 by welding, adhesive, fastener, clamp or the like so as to be removable or non-removable.

In addition to backstop 19 coupling with syringe barrel 12 and/or flange 32, backstop 19 also engages with plunger rod 16. Specifically, as depicted in FIG. 1, as backstop 19 is being slid onto syringe barrel 12 and flange 32, wings 88A and 88B are manually flexed inwardly so that feet 156A and 156B of arms 90A and 90B, respectively, are moved out of alignment with plunger rod 16. This enables backstop 19 to be fully advanced onto syringe barrel 12 without interference by arms 90A and 90B. Once backstop 19 is fully advanced onto syringe barrel 12, wings 88A and 88B are released so as to resiliently rebound back toward their unflexed state. In so doing, engagement lips 158A and 158B of arms 90A and 90B, respectively, are received within notches 87 on opposing sides of plunger rod 16 and bias against plunger rod 16. That is, wings 88A and 88B remain partially flexed when engagement lips 158A and 158B are received within notches 87 so as to produce a positive resilient force that pushes engagement lips 158 against plunger rod 16.

In this assembled configuration, syringe assembly 10 can be operated in different modes of operation. For example, when engagement lips 158 are engaging plunger rod 16, as shown in FIG. 10, engagement lips 158 and teeth 67 combine to form a ratchet that only permits plunger rod 16 to substantially move in one direction relative to backstop 19. Specifically, as previously discussed in some detail, by manually pressing down on plunger rod 16, plunger rod 16 can be advanced into compartment 30 of syringe barrel 12 by causing teeth 67 to outwardly displace and move past engagement lips 158. Such movement of plunger rod 16 can be used for dispensing dosage 168 from syringe barrel 12. However, as previously discussed, the engagement between engagement lips 158 and teeth 67 prevents plunger rod 16 from being pulled out of compartment 30.

Accordingly, backstop 19 can interact with syringe barrel 12 and plunger rod 16 to permit plunger rod 16 to advance into syringe barrel 12 but restrain plunger rod 16 from being pulled or pushed out of syringe barrel 12. Restraining the movement of plunger rod 16 out of syringe barrel 12 is helpful to prevent contamination of dosage 168 within compartment 30. For example, as depicted in FIG. 2, interior surface 22 of syringe barrel 12 proximal of stopper 14 is not sterile. Thus, if stopper 14 moves proximally within syringe barrel 12 a sufficient distance, dosage 168 within syringe barrel 12 can contact the non-sterile surface and thus become contaminated and unfit for use. This movement of stopper 14 can result from plunger rod 16 being manually pulled proximally when attached to stopper 14. Alternatively, because compartment 30 often houses a gas bubble, as discussed above, when syringe assembly 10 is subject to a reduced atmospheric pressure, such as when syringe assemblies 10 are being transported by aircraft or even when potentially transported by land but at high elevations, the gas bubble can expand in the reduced atmospheric pressure. As the gas bubble expands, it applies a force to stopper 14 trying to push stopper 14 and plunger rod 16 proximally out of syringe barrel 12. Such movement could result in dosage 168 contacting the non-sterile surface of syringe barrel 12.

Based on the above, one of the benefits of syringe assembly 10 is that backstop 19 restrains proximal movement of stopper 14 either as a result of an unwanted or unintentional exterior force being applied to plunger rod 16 or as a result of a pressure differential between compartment 30 and the surrounding environment. As a result, the probability of dosage 168 becoming contaminated is minimized.

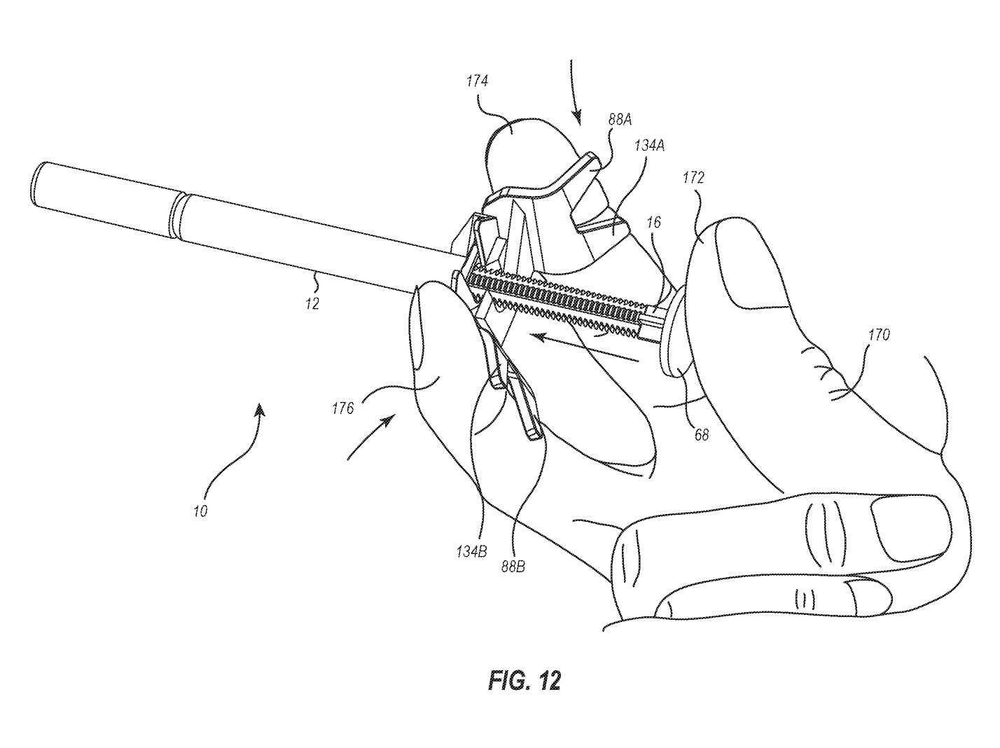

In another mode of operation, engagement lips 158 can be manually separated from teeth 67 of plunger rod 16 so that plunger rod can be freely pushed into syringe barrel 12 or freely pulled out of syringe barrel 12 without interference by teeth 67. For example, depicted in FIG. 12 is a hand 170 of an operator having a thumb 172, an index finger 174 and a middle finger 176. Syringe assembly 10 is configured so that it can be held by finger rest 134A of first wing 88A resting on top of index finger 174, finger rest 134B of second wing 88B resting on top of middle finger 174, and thumb rest 68 being disposed below thumb 172. Alternatively, finger rest 134A of first wing 88A can rest on top of middle finger 174 while finger rest 134B of second wing 88B rests on top of index finger 174.

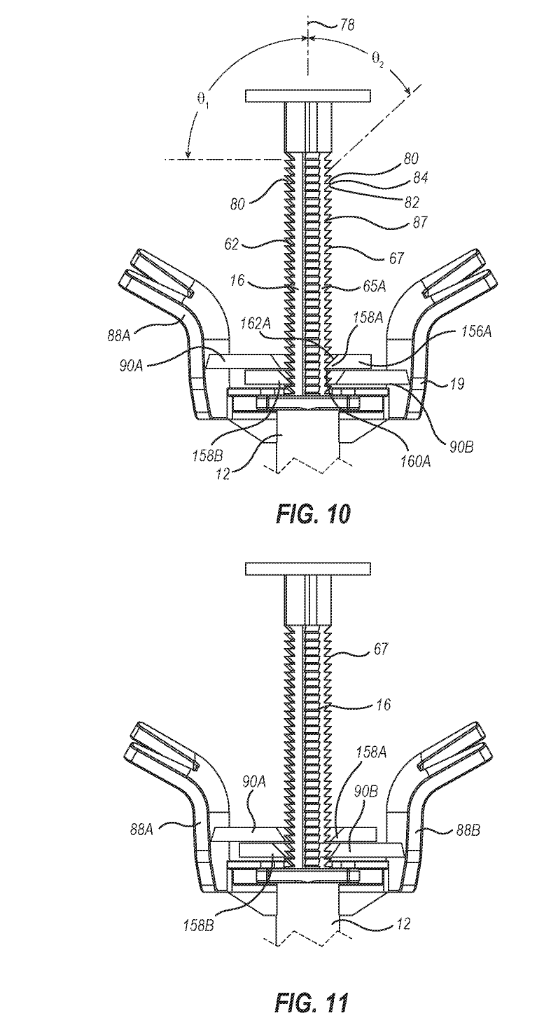

In this configuration, fingers 174 and 176 can be inwardly drawn toward each other which causes wings 88A and 88B to inwardly flex and, in turn, causes engagement lips 158A and 158B of arms 90A and 90B, respectively, to separate from teeth 67, as shown in FIG. 11 and previously discussed. That is, wings 88 and corresponding arms 90 can be selectively moved between a first position, as shown in FIG. 10, where engagement lips 158 are engaging teeth 67 to a second position, as shown in FIG. 11, where arms 90 and corresponding engagement lips 158 are spaced apart teeth 67 and corresponding plunger rod 16. With wings 88 in the second position, thumb 68 can be used to advance plunger rod 16 into syringe barrel 12 by pressing down on thumb rest 68. In so doing, syringe barrel 12 freely slides within syringe barrel 12 for dispensing dosage 168 without any interference between backstop 19 and plunger rod 16. That is, one of the benefits of the present invention is that by moving wings 88 to the second position, as shown in FIG. 11, plunger rod 16 can be smoothly and easily advanced for dispensing dosage 168 because there is no mechanical engagement between backstop 19 and plunger rod 16.

Furthermore, depressing plunger rod 16 into syringe barrel 12 while engagement lips 158 are engaging teeth 67 results in a relatively loud clicking noise as each as each tooth 67 passes over engagement lips 158. This noise can be annoying or potentially scary to young children or those who fear shots. By moving wings 88 to the second position, the clicking noise is eliminated and plunger rod 16 can be advanced for dispensing dosage 168 with substantially no noise. Once dosage 168 is dispensed, the operator can relax fingers 174 and 176 which causes wings 88 to resiliently rebound back to the first position so that engagement lips 178 again engage teeth 67 and restrict movement of plunger rod 16.

Another benefit of the present invention is that by moving wings 88 to the second position, plunger rod 16 can now be easily and quietly withdrawn a distance from within syringe barrel 12. For example, to prepare some dosages, it is necessary to draw a fluid into compartment 30 through needle 36 to mix with a component preloaded into compartment 30.

Once the liquid and component are mixed, the resulting dosage can then be dispensed by pushing plunger rod 16 into syringe barrel 12. Thus, backstop 19 provides the additional benefit that when needed, for any reason, plunger rod 16 can be withdrawn a distance within syringe barrel 12.

It is appreciated that backstop 19 may not preclude all proximal movement of plunger rod 16 and stopper 14 relative to syringe barrel 12 when wings 88 are in the first position. For example, if notches 87 between adjacent teeth 67 are larger than engagement lips 158 that are received therein, plunger rod 16/stopper 14 may be free to slide proximally and distally along the open space within the specific notch 87. In this embodiment, the permitted movement should be less than the distance that stopper 14 must move before dosage 168 can reach the non-sterile surface of barrel 12. Typically, any such free movement is less than 2 mm and more commonly less than 1 mm or less than 0.5 mm.

Furthermore, in other embodiments when wings 88 are in the first position, engagement lips 158 and teeth 67 could be configured so that when a sufficient manual force is applied to plunger rod 16, plunger rod 16 can be intentionally moved proximally as a result of lateral movement of engagement lips 158 around teeth 67. This could occur as a result of providing a slight slope to distal face 160 of engagement lips 158 and/or proximal face 80 of teeth 67, as discussed above. In this embodiment, however, the required force to move plunger rod 16 proximally needs to be greater than forces that are typically applied to plunger rod 16 that can produce unwanted proximal movement of plunger rod 16. For example, the force would need to be greater than the maximum force that stopper 14 could be subject to as a result of a change in atmospheric pressure that syringe assembly 10 is exposed to during normal transportation. Thus, in this embodiment, plunger rod 16 should only be able to move proximally when a manual pulling force greater than what could be applied to plunger rod 16 by environmental conditions is applied to plunger rod 16.

Backstop 19 also achieves other unique benefits. For example, because backstop 19 still permits plunger rod 16 to move distally for the dispensing of dosage 168, it is not necessary to remove backstop 19 after syringe assembly 10 has been transported to its final destination and it is desired to dispense dosage 168. Rather, as discussed above backstop 19 can function as an enlarged finger flange for use in gripping syringe barrel 12 during dispensing of dosage 168. Because backstop 19 functions as a finger flange, flange 32 can be formed smaller than normal during the original manufacture of syringe barrel 12, thereby decreasing the cost of syringe barrel 12. Furthermore, because some addition force is required to move plunger rod 16 distally when wings 88 are in the first position, backstop 19 also functions to help prevent unwanted dispensing of dosage 168 from syringe barrel 12.

In addition, either of wings 88 can be used to support syringe assemblies 10 in an inclined orientation on a table top or other surface for easy grasping or other manipulation. In other embodiments, backstop 19 could be removed from syringe barrel 12 prior to dispensing dosage 168. In this embodiment, flange 32 would function as a finger flange for holding syringe barrel 12 during dispensing.

Backstop 19 also has the unique benefit in that it can be used on syringe barrel 12 for any desired size of dosage 168 within syringe barrel 12. That is, independent of how far in or out plunger rod 16 is disposed within syringe barrel 12, backstop 19 can still be attached to restrain movement of plunger rod 16. Depending on the configuration, a single sized backstop 19 can also be used with a variety of syringe barrels having different configurations. Thus, a single sized backstop 19 has greater universal use than a conventional packaging case that is used to prevent movement of a plunger rod relative to a syringe barrel Likewise, backstop 19 eliminates the need to use conventional enlarged, reinforced packaging cases.

It is also appreciated that backstop 19 can have a variety of different configurations. For example, in the depicted embodiment shown in FIG. 6, mounting portion 130 of each wing 88 is secured to back end 94 of base 86. However, in other embodiments mounting portion 130 could be connected to base 86 at front end 92 or at any location between front end 92 and back end 94. Wings 88 only need to be able to flex relative to base 86 to move wings 88, and thus arms 90, between the first and second positions, as previously discussed. Where and how wings 88 are connected to base 86 can be modified accordingly. Furthermore, in the embodiment depicted, backstop 19 is shown having arms 90A and 90B projecting from wings 88A and 88B, respectively. In other embodiments, backstop 19 can be modified so as to eliminate either arm 90A or arm 90B. The remaining single arm 90 would then be used to engage plunger rod 16.

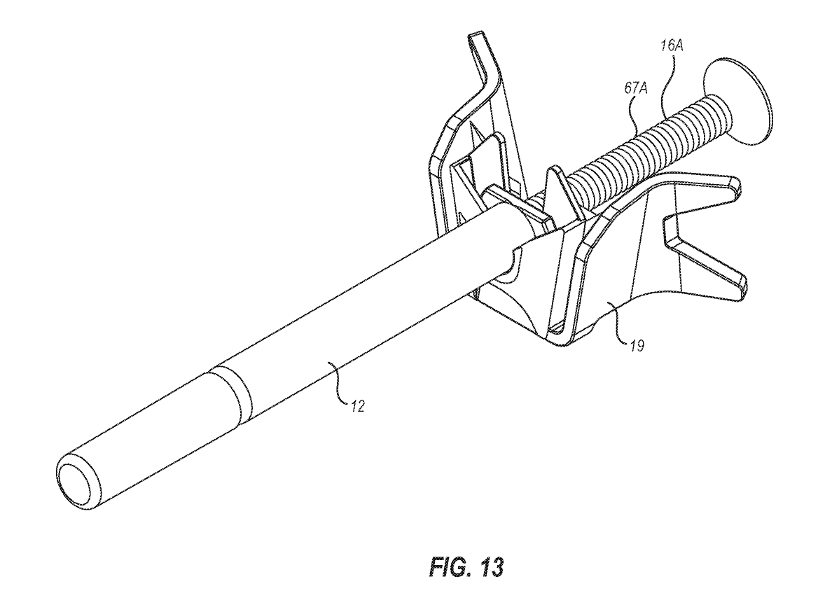

It is likewise appreciated that plunger rod 16 can also have a variety of different configurations. By way of example and not by limitation, depicted in FIG. 13 is a plunger rod 16A wherein like elements between plunger rods 16 and 16A are identified by like reference characters. Plunger rod 16 and 16A are substantially identical except that plunger rod 16A has teeth 67A that are annular and encircle shaft 62. Teeth 67A can have the same cross section, the same configuration and be used in the same way as teeth 67. Each of the plurality of teeth 67 or 67A disclosed herein can also be referred to as a rack of teeth.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A syringe assembly comprising:
a syringe barrel comprising a tubular body that bounds a compartment extending between a proximal end and an opposing distal end and a flange that radially outwardly projects from the proximal end of the body;
a stopper movably disposed with the compartment of the syringe barrel;
a plunger rod comprising a shaft having a proximal end and an opposing distal end and a plurality of teeth disposed on the shaft, the distal end of the shaft being secured to the stopper; and
a backstop comprising:
a base coupled to the syringe barrel;
a first wing and a second wing secured to the base so as to be at least partially disposed on opposing sides of the base; and
a first arm projecting from the first wing, wherein the first wing is movable between a first position where an engaging portion of the first arm engages with the plunger rod and a second position wherein the first arm is spaced apart from the plunger rod.

2. The syringe assembly as recited in claim 1, wherein the plunger rod has a first side that faces the first wing and an opposing second side that faces the second wing, the engaging portion of the first arm engaging with the second side of the plunger rod.

3. The syringe assembly as recited in claim 1, wherein the first arm has a substantially L-shaped configuration that include an elongated leg that projects from the first wing and a foot that outwardly projects from an end of the leg, the engaging portion of the first arm being disposed on the foot.

4. The syringe assembly as recited in claim 1, wherein the base has a front end and an opposing back end, the front end at least partially bounding an opening that communicates with a compartment disposed within the base, the first arm being secured to the back end of the base and projecting toward the front end of the base.

5. The syringe assembly as recited in claim 1, wherein the base comprises a top wall and a bottom wall that extend between a first side wall and an opposing second side wall and that also extend between a front end and an opposing back end of the base, the flange of the syringe barrel being at least partially disposed within a compartment disposed between the top wall and the bottom wall of the base.

6. The syringe assembly as recited in claim 5, wherein the bottom wall has a slot extending therethrough, the syringe barrel being received within the slot of the bottom wall.

7. The syringe assembly as recited in claim 6, wherein at least a portion of the slot of the bottom wall is C-shaped.

8. The syringe assembly as recited in claim 5, wherein the top wall has a slot extending therethrough, the plunger rod being received within the slot of the top wall.

9. The syringe assembly as recited in claim 5, wherein the first wing is secured to the back end of the base and projects toward the front end of the base.

10. The syringe assembly as recited in claim 5, wherein the base further comprises a back wall extending between the top wall and the bottom wall at the back end of the base, the first wing being directly secured to either the back wall or the first sidewall.

11. The syringe assembly as recited in claim 5, wherein the first wing comprises:
a mounting portion secured to the back end of the base; and
a body portion that is spaced apart from the base and extends along the base from the back end toward the front end, the body portion having an inside face that extends between a lower end disposed toward the bottom wall of the base and an upper end disposed above the top wall of the base, the first arm projecting from the inside face of the body portion of the first wing so as to be disposed above the top wall of the base.

12. The syringe assembly as recited in claim 11, wherein the first wing further comprises a first finger rest outwardly projecting from the upper end of the body portion away from the base.

13. The syringe assembly as recited in claim 1, wherein the first wing resiliently flexes as it is moved from the first position to the second position.

14. The syringe assembly as recited in claim 1, further comprising:
when the first wing is in the first position, the engagement portion of the first arm is disposed between two adjacent teeth of the plurality of teeth of the plunger rod so as to restrict movement of the plunger rod relative to the syringe barrel; and
when the first wing is in the second position, the plunger rod is freely movable relative to the syringe barrel.

15. The syringe assembly as recited in claim 1, further comprising a second arm projecting from second wing, wherein the second wing is movable between a first position where an engagement portion of the second arm engages with the plunger rod and a second position wherein the second arm is spaced apart from the plunger rod.

16. The syringe assembly as recited in claim 15, further comprising the plunger rod having a central longitudinal axis extending along the length thereof, wherein when the central longitudinal axis of the plunger rod is vertically disposed, the first arm and the second arm are overlapping with one of the first arm or second arm being vertically disposed above the other.

17. The syringe assembly as recited in claim 1, wherein the base is removably coupled to the syringe barrel.

18. The syringe assembly as recited in claim 1, wherein each of the plurality of teeth of the plunger rod radially encircle the shaft.

19. The syringe assembly as recited in claim 1, wherein the plurality of teeth of the plunger rod comprise:
a first plurality of teeth disposed longitudinally along the length of the shaft; and
a second plurality of teeth disposed longitudinally along the length of the shaft, the second plurality of teeth being radially spaced apart from the first plurality of teeth so that an elongated first slot is formed therebetween.

20. The syringe assembly as recited in claim 1, wherein when the first wing is in the second position, the plunger rod can be moved relative to the syringe barrel without contacting the backstop.

21. A method for operating a syringe, the method comprising:
inwardly flexing toward each other a first wing and a second wing of a backstop mounted on a syringe barrel so that a first arm projecting from the first wing disengages from a plunger rod projecting into a compartment of the syringe barrel;
moving the plunger rod relative to the syringe barrel while the first wing and the second wing are inwardly flexing; and
allowing the first wing and the second wing to resiliently rebound after moving the plunger rod so that the first arm engages with the plunger rod.

22. The method as recited in claim 21, wherein the step of inwardly flexing the first wing and the second wing causes a second arm projecting from the second wing to disengage from the plunger rod.

23. The method as recited in claim 21, wherein the step of inwardly flexing the first wing and the second wing comprises a hand of an operator engaging the first wing with an index finger and engaging the second wing with a middle finger, the middle finger and the index finger being drawn toward each other to inwardly flex the first wing and the second wing.

24. The method as recited in claim 21, wherein the step of moving the plunger rod comprises advancing the plunger rod a distance into the compartment of the syringe barrel.

25. The method as recited in claim 21, wherein the step of moving the plunger rod comprises retracting a portion of the plunger rod out of the compartment of the syringe barrel.

26. The method as recited in claim 21, wherein the plunger rod moves without contacting the backstop.
